(19) United States
(12) Patent Application Publication
(10) Pub. No.: US 2020/0330467 A1
(43) Pub. Date: Oct. 22, 2020

(54) METHOD AND PHARMACEUTICAL COMPOSITIONS FOR THE TREATMENT OF MULTIPLE MYELOMA

(71) Applicants: INSERM (INSTITUTE NATIONAL DE LA SANTÉ ET DE LA RECHERCHE MÉDICALE), Paris (FR); UNIVERSITÉ DE MONTPELLIER, Montpellier (FR); INSTITUT RÉGIONAL DU CANCER DE MONTPELLIER, Montpellier (FR); CENTRE HOSPITALIER UNIVERSITAIRE DE MONTPELLIER, Montpellier (FR); CENTRE NATIONAL DE LA RECHERCHE SCIENTIFIQUE (CNRS), Paris (FR)

(72) Inventors: Jérôme MOREAUX, Montpellier Cedex 5 (FR); Eric JULIEN, Montpellier Cedex 5 (FR); Fanny IZARD, Montpellier Cedex 5 (FR); Charlotte GRIMAUD, Montpellier Cedex 5 (FR); Laurie HERIOU, Montpellier Cedex 5 (FR)

(21) Appl. No.: 15/998,657

(22) PCT Filed: Feb. 15, 2017
(86) PCT No.: PCT/EP2017/053435

§ 371 Date: Aug. 16, 2018

(30) Foreign Application Priority Data
Feb. 16, 2016 (EP) 16305176.6

(51) Int. Cl. A61K 31/517; A61K 31/192; A61P 35/00

(52) U.S. Cl. CPC A61K 31/517; A61K 45/06; A61P 35/00; A61K 31/192

(57) ABSTRACT

The present invention relates to methods and pharmaceutical compositions for the treatment of multiple myeloma. In particular, the present invention relates to a method of treating multiple myeloma in a patient in need thereof comprising administering to the patient a therapeutically effective amount of a SETD8 inhibitor.

### FIELD OF THE INVENTION

The present invention relates to methods and pharmaceutical compositions for the treatment of multiple myeloma.

### BACKGROUND OF THE INVENTION

Multiple Myeloma (MM) is a currently incurable malignant plasma cell disease, with 25,000 new patients per year in the EU and a median survival of five to six years¹. This disease develops primarily in the bone marrow and is associated with end organ damages: bone lesions due to abnormal bone turnover induced by tumor cells, renal failure due to high levels of monoclonal immunoglobulin (Ig), anemia due to bone marrow invasion by tumor cells. Malignant plasma cells (Multiple Myeloma Cells, MMCs) are clonal cells that exhibit the same rearranged heavy and light chain Ig genes, with somatic mutations in the variable domains of Ig genes that are fixed throughout disease course. For virtually all patients, Multiple Myeloma (MM) disease is preceded by a premalignant phase, called monoclonal gammopathy of undetermined significance (MGUS), characterized by the accumulation in the bone marrow of clonal plasma cells surviving for years in the bone marrow and the detection of a clonal Ig in the serum. MGUS frequency increased rapidly with age and occurs in 5.7% the population older than 70 years. Premalignant plasma cells are in an oncogene-induced senescence state, with deregulation of oncogenes (Cyclin D1, Ras and growth factors), partial inactivation of the Rb pathway and accumulation of p21 and p16. MM disease arises at a 1% rate per year in patients with MGUS, independently of the duration of premalignant phase.

Treatment of MM¹ consists of (1) an induction phase with four monthly courses of high doses of corticosteroid (Dexamethasone) and a proteasome inhibitor (Velcade) in association with a cell cycle targeting drug or an immunomodulatory drug, (2) a short-term exposure to high dose of Melphalan, an alkylating agent followed with autologous stem cell transplantation to rescue hematopoiesis and (3) a maintenance treatment with dexamethasone and an immunomodulatory drug (Thalidomide or Revlimid), which targets Cereblon, an F-box protein of the CUL4-DDB1 ubiquitin ligase complex. These regimens do not cure patients and MM repeatedly relapses until the patient succumbs to the disease.

The molecular events governing the onset and progression of malignant transformation are triggered by DNA alterations (translocations, amplifications or deletions, mutations) and defects in pattern of epigenetic modifications in chromatin³, including changes in DNA methylation and in histone methylation and acetylation. These epigenetic changes are often critical in the initiation and progression of many cancers⁴. In multiple myeloma (MM), the profile of DNA methylation comprises genomic global hypomethylation and promoter hypermethylation of tumor suppressor genes^{5,6}. More recently, hypermethylation of GPX3, RBP1, SPARC and TGFBI genes was demonstrated to be associated with significantly shorter overall survival, independent of age, ISS score and adverse cytogenetics⁷. Thus, clinical trials for MM treatment are ongoing with DNMT inhibitors (DNMTi) as monotherapy or combined with lenalidomide or dexamethasone⁸. Histone deacetylases (HDAC) represent another molecular targets for the treatment of different cancers including MM⁹⁻¹². Romidepsin and Vorinostat (SAHA) have been approved by the Food and Drug Administration (FDA) for the treatment of cutaneous T-cell lymphoma¹³ and several HDAC inhibitors (HDACi) are evaluated in clinical trials in MM^{8,9}. In this regard, combination of panobinostat-bortezomib-dexamethasone (PANORAMA) and of vorinostat-bortezomib (VANTAGE 088) have been initiated in two large phase III clinical trials^{14,15}. Results of VANTAGE 088 trial showed that association of vorinostat and bortezomib prolonged significantly progression free survival in patients with relapsed or refractory MM¹⁵. In PANORAMA clinical trial, panobinostat pan-HDACi treatment combined with bortezomib and dexamethasone resulted in a significant progression-free survival improvement in patients with relapsed MM¹⁶. However, identification of biomarkers predictive for sensitivity of MMCs to epigenetic therapies remains an important objective to improve clinical trials. We recently reported gene expression (GEP)-based risk scores to predict the sensitivity of MMC to DNMTi^{17,18} and HDACi¹⁷. These scores allow the identification of MM patients who could benefit from HDACi or DNMTi treatment.

SETD8 (also known as SET8, PR-Set7, KMT5A) is the sole enzyme responsible for the monomethylation of histone H4 at lysine 20 (H4K20me1) which has been linked to chromatin compaction and cell-cycle regulation¹⁹⁻²¹. In addition, SETD8 also induces the methylation of non-histone proteins, such as the replication factor PCNA and the tumor suppressor P53 and its stabilizing protein Numb^{22,23,24}. While SETD8-mediated methylation of P53 and Numb inhibits apoptosis, PCNA methylation upon SETD8 enhances the interaction with the Flap endonuclease FEN1 and promotes cancer cell proliferation^{23,24}. Consistent with this, overexpression of SETD8 is found in various types of cancer and has been directly implicated in breast cancer invasiveness and metastasis²⁵. A role of SDT8 in development of Multiple Meyloma is not known, however.

### SUMMARY OF THE INVENTION

The present invention relates to methods and pharmaceutical compositions for the treatment of multiple myeloma. In particular, the present invention is defined by the claims.

### DETAILED DESCRIPTION OF THE INVENTION

In order to identify new epigenetic targets for MM cancer treatment, the inventors asked which genes encoding epigenetic factors are differentially expressed between normal bone marrow plasma cells (BMPCs, n=7), MM plasma cells from newly diagnosed patients (MMCs, n=206) and human myeloma cell lines (HMCLs, N=40). They identified a significant overexpression of the histone methyltransferase SETD8 in HMCLs compared to normal BMPCs and primary MMCs from newly diagnosed patients. Here, the inventors provide evidence that SETD8 overexpression is an adverse prognosis factor in multiple myeloma. The inhibition of this epigenetic enzyme by the chemical drug UNC0379 causes SETD8 degradation and H4K20me1 depletion, which leads to cell-cycle defects and apoptosis in U2OS and human myeloma cell lines. Remarkably, treatment of MM patient samples with UNC 0379 leads to reduce the percentage of meyloma cells without significant toxicity on the non-myeloma cells, suggesting a specific addiction of primary myeloma cells to the SETD8 activity. Finally, combining low dose of UNC0379 with melphalan strongly enhances the appearance of DNA damage, suggesting that SETD8 inhibition represent a promising strategy to improve conventional treatment of multiple myeloma.

Accordingly a first object of the present invention relates to a method of treating multiple myeloma in a patient in need thereof comprising administering to the patient a therapeutically effective amount of a SETD8 inhibitor.

The term “multiple myeloma” as used herein refers to a disorder characterized by malignant proliferation of plasma cells derived from a single clone. It is diagnosed using standard diagnostic criteria. Typically, low red blood cell count and/or elevated protein levels in the blood or protein in the urine is an early indicator; a bone marrow biopsy showing high levels of myeloma cells (>10% plasma cells) in the bone marrow is more definitive. The presence of the M protein in the serum and/or presence of lytic lesions in the bones are also diagnostic indicators of the disorder.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “SETD8” has its general meaning in the art and refers to the sole enzyme responsible for the monomethylation of histone H4 at lysine 20 (H4K20me1) which has been linked to chromatin compaction and cell-cycle regulation¹⁹⁻²¹. SETD8 is also known as SETS, PR-Set7, KMT5A. Accordingly a “STED8 inhibitor” refers to any compound that is able to inhibit the activity or expression of SETD8. In particular the SETD8 inhibitor of the present invention is a compound that is able to inhibit the catalytic activity of the enzyme i.e. the monomethylation of histone H4 at lysine 20.

SETD8 inhibitors are well known in the art and typically include the compounds described in J Med Chem. 2014 Aug. 14; 57(15):6822-33. In some embodiments, the SETD8 inhibitor is the nahuoic acid A that has the formula of

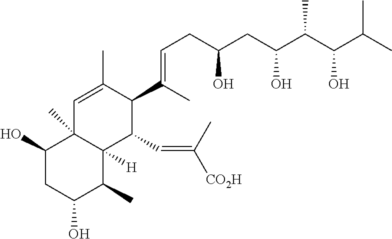 [SMILES: CC1=C[C@@]2(C)[C@H]([C@@H](C)[C@H](O)C[C@H]2O)[C@@H](/C=C(\C)C(=O)O)[C@@H]1/C(C)=C/C[C@H](O)C[C@@H](O)[C@H](C)[C@@H](O)C(C)C]

In some embodiments, the SETD8 inhibitor is the UNC0379 small-molecule inhibitor that has the formula of:

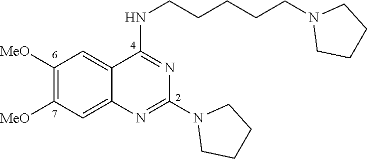 [SMILES: COc1cc2nc(N3CCCC3)nc(NCCCCCN3CCCC3)c2cc1OC]

In some embodiments, the SETD8 inhibitor is an inhibitor of SETD8 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of SETD8 mRNA by binding thereto and thus preventing protein translation or increasing mRNA degradation, thus decreasing the level of SETD8, and thus activity, in a cell. For example, antisense oligonucleotides of at least about 15 bases and complementary to unique regions of the mRNA transcript sequence encoding SETD8 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. SETD8 gene expression can be reduced by contacting a patient or cell with a small double stranded RNA (dsRNA), or a vector or construct causing the production of a small double stranded RNA, such that SETD8 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing SETD8. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the SETD8 inhibitor of the present invention is administered to the patient in combination with a chemotherapeutic agent. As used herein, the term “chemotherapeutic agent” refers to any compound that can be used in the treatment, management or amelioration of cancer, including peritoneal carcinomatosis, or the amelioration or relief of one or more symptoms of a cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azascrine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycins, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′2″-trichlorotriethylamine; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; etoposide (VP-16); ifosfamide; mitomycin C; mitoxantrone; vinblastine; vincristine; vinorelbine; navelbine; novantrone; teniposide; daunomycin; aminopterin; xeloda; ibandronate; difluoromethylornithine (DMFO); retinoic acid; esperamicins; capecitabine; imexon; tyrosine kinase inhibitors, such as epidermal growth factor receptor tyrosine kinase inhibitor erlotinib; proteasome inhibitors such as bortezomib thalidomide, lenalidomide corticosteroids such as prednisone and dexamethasone (Decadron®). pomalidomide, the keto-epoxide tetrapeptide proteasome carfilzomib, the anti-CS-1 antibody elotuzumab, and histone deacetylase inhibitors of vorinostat and panabinostatand pharmaceutically acceptable salts, acids or derivatives of any of the above.

A further object of the present invention relates to a method for predicting the survival time of a patient suffering from multiple myeloma comprising i) determining the expression level of SETD8 in a sample of multiple myeloma cells obtained from the patient, ii) comparing the expression level determined at step i) with a predetermined reference value and iii) concluding that the patient will have a short survival time when the level determined at step i) is higher than the predetermined reference value or concluding that the patient will have a long survival time when the expression level determined at step i) is lower than the predetermined reference value.

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

In some embodiments, the expression level of SETD8 is determined by determining the quantity of STD8 mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization. Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

In some embodiments, the expression level of SETD8 is determined at the protein level. Typically, the sample is contacted with a binding agent specific for SETD8 (e.g. an antibody). For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of SETD8 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of SETD8 in a group of reference, one can use algorithmic analysis for the statistic treatment of the measured expression levels of SETD8 in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples of multiple myeloma cells; b) providing for each ample provided at step a), information relating to the actual clinical outcome for the corresponding patient (i.e. the duration of the survival); c) providing a serial of arbitrary quantification values; d) determining the expression level of SETD8 for each sample contained in the collection provided at step a); e) classifying said samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the patients from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g). For example the expression level of SETD8 has been assessed for 100 samples of 100 patients. The 100 samples are ranked according to the expression level of SETD8. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patient, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of SETD8 corresponding to the boundary between both subsets for which the p value is minimum is considered as the predetermined reference value. It should be noted that the predetermined reference value is not necessarily the median value of expression levels of SETD8 Thus in some embodiments, the predetermined reference value thus allows discrimination between a poor and a good prognosis for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of SETD8 with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the expression level of SETD8, where values higher than 5 reveal a poor prognosis and values less than 5 reveal a good prognosis. In some embodiments, a patient may be assessed by comparing values obtained by measuring the expression level of SETD8 and comparing the values on a scale, where values above the range of 4-6 indicate a poor prognosis and values below the range of 4-6 indicate a good prognosis, with values falling within the range of 4-6 indicating an intermediate occurrence (or prognosis).

According to the present invention the therapeutically effective amount is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

A further object of the present invention relates to a method of treating multiple myeloma in a subject in need thereof comprising i) determining the survival time of the patient by the method as above described and ii) administering to the patient a therapeutically effective amount of a SETD8 inhibitor when it is concluded that the patient will have a short survival time.

According to the invention, the SETD8 inhibitor is administered to the patient in the form of a pharmaceutical composition. Typically, the SETD8 inhibitor may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The SETD8 inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the patient being treated. The person responsible for administration will, in any event, determine the appropriate dose for the individual patient.

The invention will be further illustrated by the following figures and examples. However, these examples and figures should not be interpreted in any way as limiting the scope of the present invention.

### FIGURES

FIG. 1: SETD8 expression in MM. A. SETD8 gene expression in BMPCs, patients' MMCs and HMCLs. Data are MASS-normalized Affymetrix signals (U133 plus 2.0 microarrays). Statistical difference was assayed using a t-test.

FIG. 2: SETD8 gene expression in the 8 groups of the UAMS molecular classification of multiple myeloma. Gene expression profiling of MMCs of the patients of UAMS-TT2 cohort were used. PR: proliferation, LB: low bone disease, MS: MMSET, HY: hyperdiploid, CD1: Cyclin D1-Cyclin D3, CD2: Cyclin D1-Cyclin D3, MF: MAF, MY: myeloid.

FIG. 3: Prognostic value of SETD8 expression in MM. (A) Lower overall survival of patients of UAMS-TT2 cohort (n=256) whose MMCs highly expressed SETD8 gene. The splitting of the patients into two groups according to SETD8 expression in MMCs was done using the Maxstat algorithm. (B) Lower overall survival of patients of the UAMS-TT3 cohort (n=158) whose MMCs highly expressed RECQ1 gene.

FIG. 4: SETD8 is significantly overexpressed in MM patients with ch 1q21 gain and high growth proliferation index. (A) Association between SETD8 expression and patients' genetic abnormalities was completed using UAMS-TT2 cohort. (B) SETD8 expression in the different gene expression-based proliferation index groups (GPI).

FIG. 5: UNC0379 treatment causes SETD8 and H4K20me1 depletion followed by DNA damage and checkpoint activation in U2OS cells. (A) Immunoblot analysis with anti-SETD8 and anti-H4K20me1 and anti-histone H4 (loading control) of U2OS cells trated with DMSO or with 10 μM of UNC0379 for 24 h. (B) Immunoblot analysis with anti-SETD8 and anti-tubulin (loading control) of DMSO treated cells or UNC0379 treated cells in presence or not of MG123 (proteasome inhibitor). (C) immunoblot analysis of whole-cell extracts with anti-tubulin (loading control), anti-phospho/CHK1 (marker of DNA damage checkpoint activation), anti-P53, anti-P21, anti-γH2A.X (marker of DNA damage) in cells treated with DMSO or UNC0379 during 24 hours.

FIG. 6: UNC0379 treatment induces myeloma cell growth inhibition, H4K20me1 depletion and DNA damages. (A) UNC0379 induces a dose-dependent inhibition of cell growth in HMCL. HMCL were cultured for 4 days in 96-well flat-bottom microtitre plates in RPMI 1640 medium, 10% fetal calf serum, 2 ng/ml interleukin six culture medium (control), and graded concentrations of UNC0379. At day 4 of culture, the viability was assessed by CellTiter-Glo® Luminescent Cell Viability Assay. The IC50 (concentration responsible for 50% of the maximal inhibitory effect), was determined using GraphPad PRISM software. Data are mean values±standard deviation (SD) of five experiments determined on sextuplet culture wells; (B) immunoblot analysis of whole-cell extracts with anti-tubulin (loading control), anti-SETD8, anti-histone H4 (loading control), anti-H4K20me1 and anti-γH2A.X in XG1, XG7 and XG12 HMCLs treated 24 hours with DMSO or UNC0379 at 5 μM.

FIG. 7: UNC0379 treatment results in MM cell apoptosis induction. Apoptosis induction was analyzed with Annexin V APC staining by flow cytometry. The shown data is one representative experiment and mean values±SD of 3 separate experiments. Statistical analysis was done with a paired t-test.

FIG. 8: UNC0379 synergizes with melphalan to induce DNA damage response in MM cells. 53BP1 staining was used as marker for DNA damage. The number of 53BP1 foci found in each cell was counted, three days after doxycycline treatment. At least 300 cells were counted for each treatment group. The percentage of cells with 53BP1 foci per cell is displayed in the histograms.

FIG. 9: UNC0379 induces mortality of primary MM cells from patients. Mononuclear cells from 8 patients with MM were treated with different doses of UNC0379. At day 4 of culture, the viability and total cell counts were assessed and the percentage of CD138 viable plasma cells was determined by flow cytometry. Results are median values of the numbers of myeloma cells in the culture wells. The values were compared with a Wilcoxon test for pairs.

### EXAMPLE

Material & Methods

Human Myeloma Cell Lines (HMCLs) and primary multiple myeloma cells of patients. Human myeloma cell lines HMCLs, N=40 were obtained as previously described or purchased from DSMZ and American Type Culture Collection. Microarray data are deposited in the ArrayExpress public database (accession numbers E-TABM-937 and E-TABM-1088). Patients presenting with previously untreated multiple myeloma (N=206) or monoclonal gammopathy of undetermined significance (N=5) at the university hospitals of Heidelberg and Montpellier as well as 7 healthy donors have been included in the study approved by the ethics committee of Montpellier and Heidelberg after written informed consent in accordance with the Declaration of Helsinki. Clinical parameters and treatment regimens of the MM patients included in the Heidelberg-Montpellier (HM) cohort were previously described²⁶. Gene expression profiling (GEP) of purified MMCs was assayed using Affymetrix U133 2.0 plus microarrays (Affymetrix, Santa Clara, Calif., USA) as described²⁷ and data normalized using the MASS Affymetrix algorithm. The .CEL and MASS files are deposited in the ArrayExpress public database (http://www.ebi.ac.uk/arrayexpress/), under accession number E-MTAB-362. We also used publicly available MASS normalized GEP data (GEO, http://www.ncbi.nlm.nih.gov/geo/, accession number GSE2658) from purified MMCs of a cohort of 345 patients treated with total therapy 2 protocol (UAMS-TT2 cohort) at the University of Arkansas for Medical Sciences (UAMS, Little Rock, USA)²⁸. T(4; 14) translocation was evaluated using MMSET spike expression²⁹ and del17p13 surrogated by TP53 probe set signal³⁰ for UAMS-TT2 patients. Gene expression data of normal memory B cells (MB), preplasmasts, plasmablasts and early plasma cells^{31,32} are deposited in the ArrayExpress databases under accession numbers E-MEXP-2360 and E-MEXP-3034.

Immunoblot Analysis

For immunoblot analysis, cells washed with phosphate-buffered saline (PBS) were lysed in Laemmli buffer. After measuring protein quantity by Bradford, equal amounts of protein were resolved by SDS-PAGE, transferred to a nitrocellulose membrane and probed with one of the following antibodies: anti-SETD8 (1:1000, Cell Signaling Technology), anti-H4K20me1 (1:1000 cell signaling) anti-phospho-H2A.X-Ser139 (1:1000, millipore). Membranes were then incubated with the appropriate horseradish peroxidase (HRP)-conjugated secondary antibodies. The immunoreactive bands were detected by chemiluminescence.

Sensitivity of HMCLs to SET8 Inhibitor.

HMCL were cultured with graded concentrations of the SETD8 inhibitor UNC0379³³.HMCL cell growth was quantified with a Cell Titer Glo Luminescent Assay (Promega, Madison, Wis., USA) and the 50% inhibitory concentration (IC₅₀) was determined using GraphPad Prism software (http://www.graphpad.com/scientific-software/prism/)^{17,18,34}.

Sensitivity of Primary Myeloma Cells to SETD8 Inhibitor.

Primary myeloma cells of 6 patients were cultured with or without graded concentrations of UNC0379 SETD8 inhibitor. MMCs cytotoxicity was evaluated using anti-CD138-PE mAb (Immunotech, Marseille, France) as described^{18,35}. Results were analyzed using GraphPad Prism (http://www.graphpad.com/scientific-software/prism/).

DNA Repair Foci—Immunofluorescence Microscopy

After deposition on slides using a Cytospin centrifuge, cells were fixed with 4% PFA, permeabilized with 0.5% Triton in PBS and saturated with 5% bovine milk in PBS. The rabbit anti-53BP1 antibody (clone NB100-304, Novus Biologicals, Cambridge, United Kingdom) and the mouse anti-γH2AX (Ser139) antibody (clone JBW301, Merck Millipore, Darmstadt, Germany) were diluted 1/300 and 1/100 respectively in 5% bovine milk in PBS, and deposited on cytospins for 90 minutes at room temperature. Slides were washed twice and antibodies to rabbit or mouse immunoglobulins conjugated to alexa 488 (diluted 1/500 in 5% bovine milk in PBS) were added for 45 minutes at room temperature. Slides were washed and mounted with Vectashield and 1% DAPI. Images and fluorescence were captured with a ZEISS Axio Imager Z2 microscope (X63 objective) (company, town state), analyzed with Metafer (version3.6, company, town state) and ImageJ softwares. The number of 53BP1 and γH2AX foci was counted in at least 300 nuclei.

Statistical Analysis

Gene expression data were analyzed using SAM (Significance Analysis of Microarrays) software³⁶ as published²⁹. The statistical significance of differences in overall survival between groups of patients was calculated by the log-rank test. Multivariate analysis was performed using the Cox proportional hazards model. Survival curves were plotted using the Kaplan-Meier method. All these analyses have been done with R.2.10.1 (http://www.r-project.org/) and bioconductor version 2.5. Significantly enriched pathways were identified using Reactome functional interaction map. Gene set enrichment analysis was carried out by computing overlaps with canonical pathways and gene ontology gene sets obtained from the Broad Institute³⁷.

Results

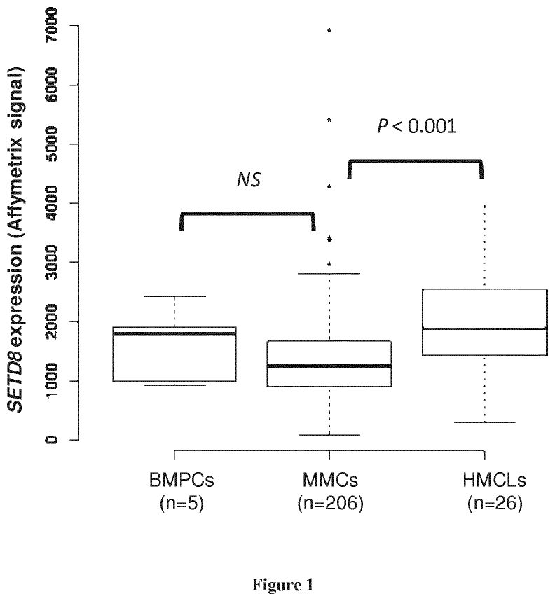

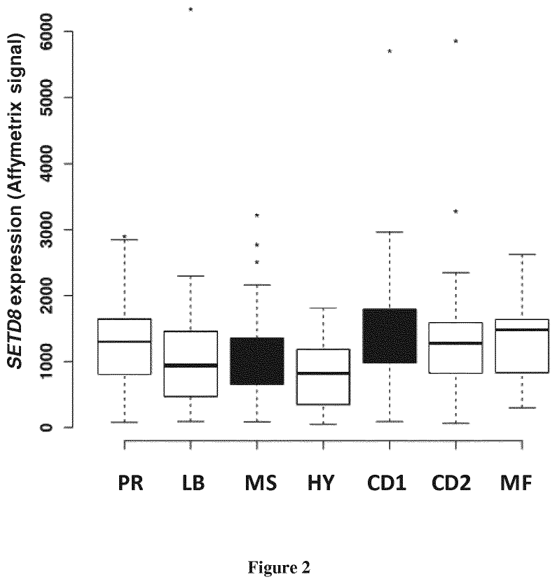

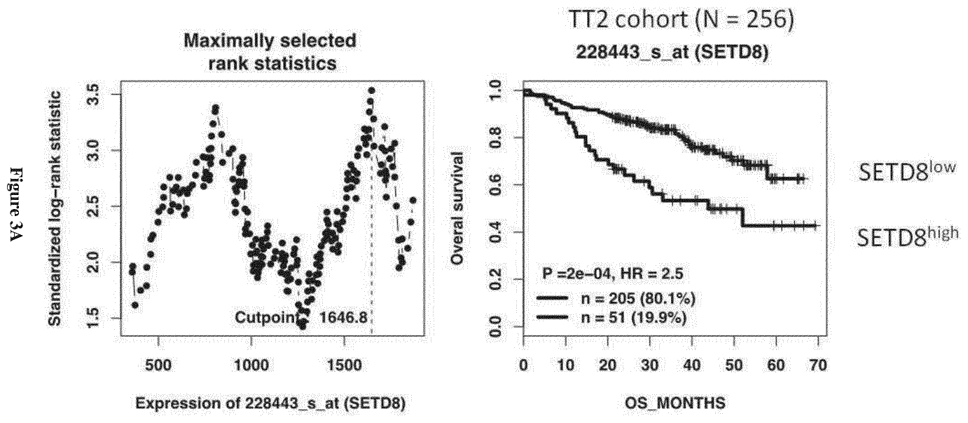

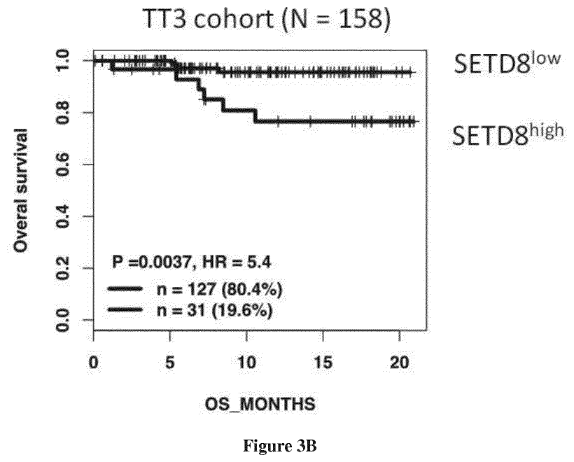

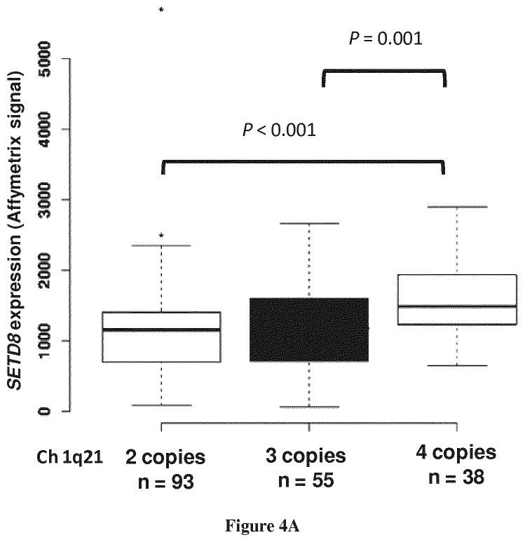

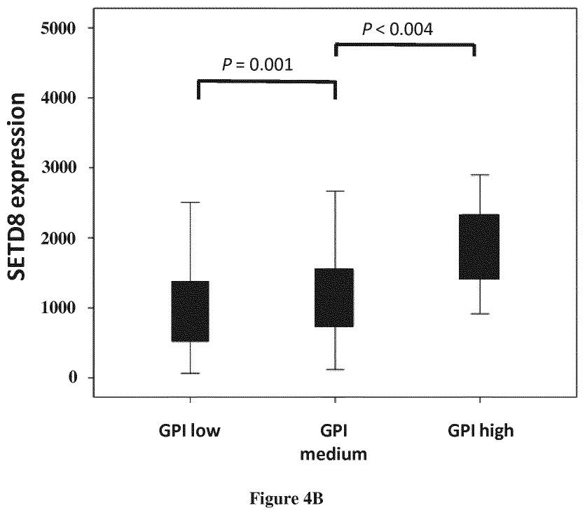

As shown in FIG. 1, SETD8 gene was not significantly differentially expressed between multiple myeloma cells (MMCs) of patients (median 1253, range 92-6928) compared to normal bone marrow plasma cells (BMPCs) (median=1798; range: 922-2422)(P=NS). However, we noticed some abnormal spiked expression in several patients (P<0.001) and a relative higher expression in human myeloma cell lines (HMCLs) (median 1880, range 286-3960) compared to primary MMCs or BMPCs (P<0.001) (FIG. 1). Primary MMCs of previously untreated patients can be classified into seven molecular groups associated with different patients' survival³⁸. SETD8 expression was not significantly deregulated in a specific subgroup of the molecular classification of MM (FIG. 2). Nevertheless, a high SETD8 expression, defined using maxstat R package²⁹, coincided with shorter overall survival (OS) in two independent cohorts of newly-diagnosed MM patients (P=2E-4 in the UAMS-TT2 cohort (N=256) and P=0.003 in the UAMS-TT3 cohort (N=158)) (FIG. 3). In addition, a significant overexpression of SETD8 is observed in MM patients with 4 copies of chromosome regions 1q21, which are linked to a poor prognosis (FIG. 4A). No significant overexpression of SETD8 was identified in patients with deletion 17p or t(4; 14) translocation. There is a significant gradual increase in SETD8 expression from patients with a low growth proliferation index (GPI)²⁶ to GPI^{medium} and GPI^{high} groups (FIG. 4B). Taken together, these results suggest that SETD8 overexpression is associated with a prognostic value in MM patients.

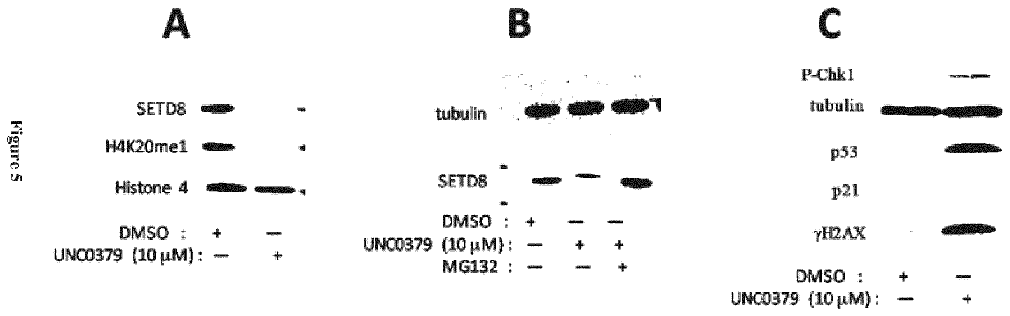

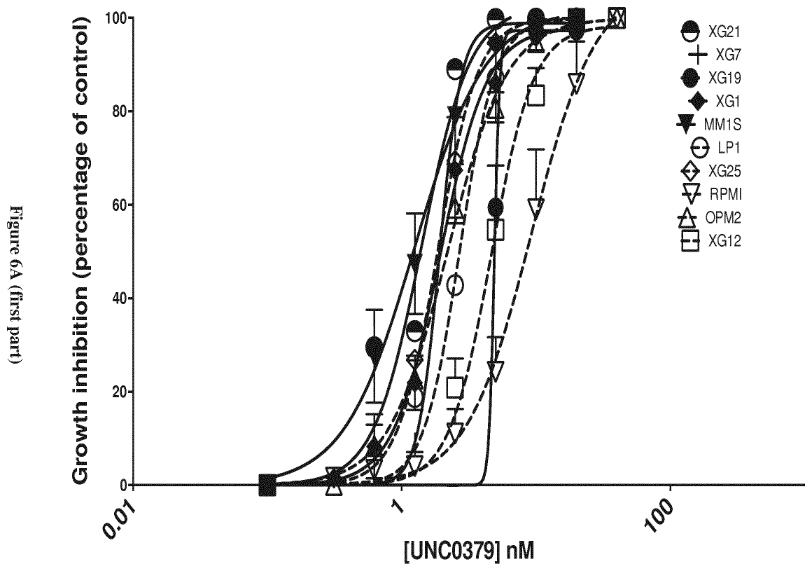

Given the role of SETD8 in the control of genome integrity and cell proliferation, we investigated the interest of the SETD8 inhibitor, UNC0379, to eradicate MM cells. UNC0379 treatment is efficient to decrease the levels of H4K20me1 within 24 hours, whereas no significant change was observed for other methylated histone marks such as H3K9me1 and H3K27me1 at this time in the osteaosarcoma U2OS cell line (FIG. 5A). Interestingly, UNC0379 treatment also caused the proteolytic degradation of SETD8, which was prevented by the inhibition of the 26S proteasome pathway by the chemical proteasome inhibitor MG132 (FIG. 5B). Since SETD8 is targeted for degradation in response to DNA damage²⁰, the degradation of SETD8 upon UNC0379 might be related the appearance of DNA damage and the activation of the DNA damage checkpoint, as observed by the increase in γH2A.X and the phosphorylation of CHK1 in UNC0379-treated cells (FIG. 5C). Consistent with this, UNC0379 treatment led to the stabilization of P53 and an increase in the levels of the cell-cycle inhibitor P21 (FIG. 5C). These results indicate that UNC0379 can penetrate into human cells and induce a specific inhibition of SETD8 activity followed by its proteolytic degradation via the proteasome.

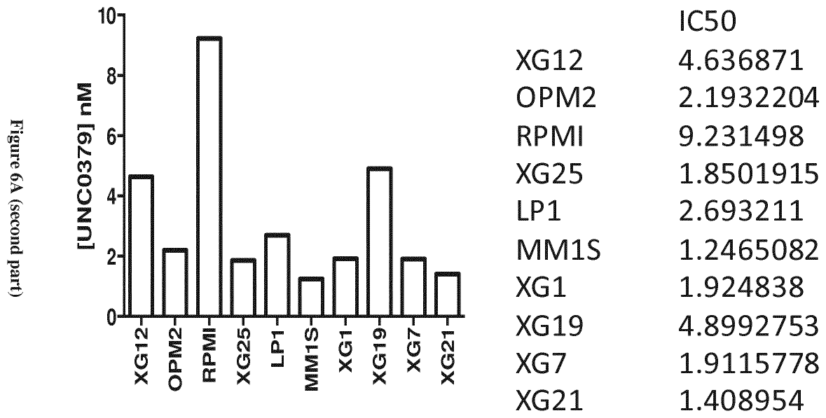

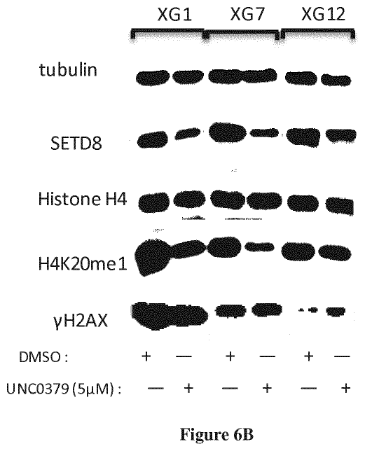

The effect of UNC0379 was examined in 10 different human myeloma cell lines representative of the patients' molecular heterogeneity³⁹. As shown in FIG. 6, UNC0379 induced a dose dependent inhibition of cell growth in all investigated HMCLs with a median IC50 of 2.05 μM (range: 1.24-9.23 μM) (FIG. 6A). As described in FIG. 5 for U2OS cells, this cell-growth inhibition in HMCLs resulted from a decrease in levels and activity of SETD8 upon UNC0379 treatment (FIG. 6B).

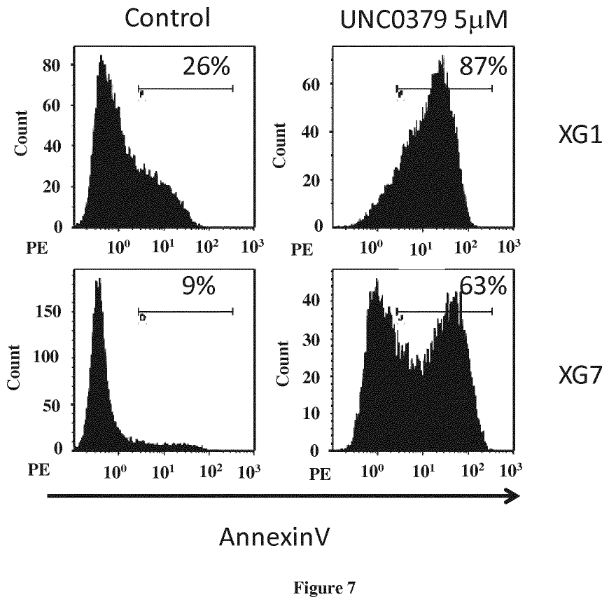

By flow cytometry experiments, we showed that UNC0379 (5 mM)-mediated SETD8 inhibition is followed by apoptosis in XG1 and XG7 HMCLs with 87%, 63% of annexin-V positive respectively (FIG. 7, P<0.05).

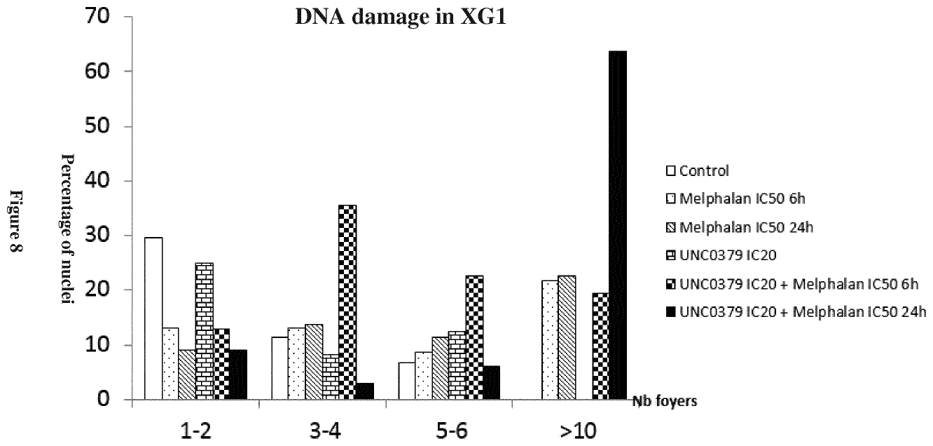

These results allowed us to examine the combination of a sub-lethal dose of UNC0379 with genotoxic drugs currently used in MM treatment. UNC0379 significantly synergizes with melphalan to induce accumulation of DNA double strand breaks, as evidenced by increased accumulation of 53BP1 foci (FIG. 8, P<0.001, and data not shown).

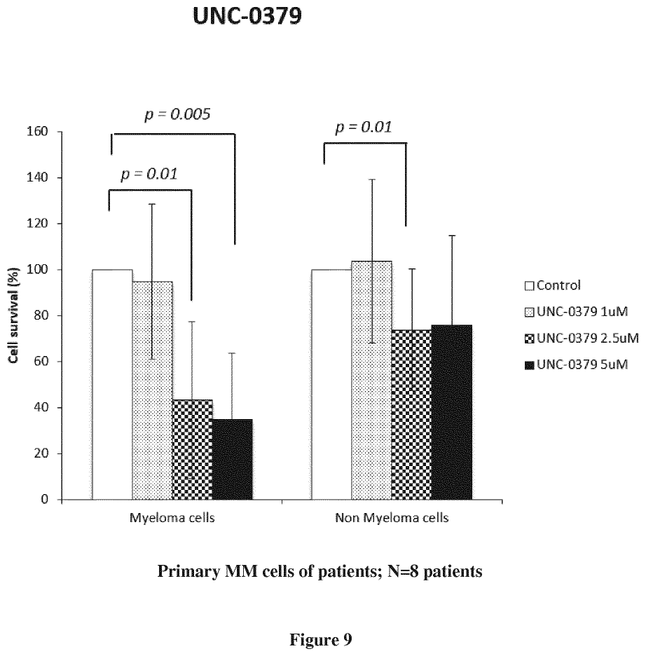

We next tested the impact of UNC0379 treatment on primary myeloma cells derived from 8 distinct patients. As shown in FIG. 9, UNC0379 induced a significant apoptosis of primary myeloma cells from patients co-cultured with their bone marrow environment and recombinant IL-6¹⁷ (n=8). Strikingly, SETD8 inhibitor significantly reduced the median number of viable myeloma cells by 19%, 57% and 66% at respectively 1, 2.5 and 5 μM (P=NS, P=0.01 and P=0.005 respectively; n=8) (FIG. 9) without significant toxicity on the non-myeloma cells (FIG. 9). These data demonstrated a specific toxicity of UNC0379 on myeloma cells.

Taken together, these data underline the therapeutic interest of SETD8 inhibitor in MM and especially in patients characterized by high SETD8 expression and a poor prognosis.

### REFERENCES

Throughout this application, various references describe the state of the art to which this invention pertains. The disclosures of these references are hereby incorporated by reference into the present disclosure.
1. Rajkumar S V. Treatment of multiple myeloma. Nat Rev Clin Oncol. 2011; 8(8):479-491.
2. Landgren O, Kyle R A, Pfeiffer R M, et al. Monoclonal gammopathy of undetermined significance (MGUS) consistently precedes multiple myeloma: a prospective study. Blood. 2009; 113(22):5412-5417.
3. Baylin S B. DNA methylation and gene silencing in cancer. Nat Clin Pract Oncol. 2005; 2 Suppl 1:S4-11.
4. Kondo Y. Epigenetic cross-talk between DNA methylation and histone modifications in human cancers. Yonsei Med J. 2009; 50(4):455-463.
5. Heuck C J, Mehta J, Bhagat T, et al. Myeloma is characterized by stage-specific alterations in DNA methylation that occur early during myelomagenesis. J Immunol. 2013; 190(6):2966-2975.
6. Walker B A, Wardell C P, Chiecchio L, et al. Aberrant global methylation patterns affect the molecular pathogenesis and prognosis of multiple myeloma. Blood. 2010.
7. Kaiser M F, Johnson D C, Wu P, et al. Global methylation analysis identifies prognostically important epigenetically inactivated tumour suppressor genes in multiple myeloma. Blood. 2013.
8. Maes K, Menu E, Van Valckenborgh E, Van Riet I, Vanderkerken K, De Bruyne E. Epigenetic Modulating Agents as a New Therapeutic Approach in Multiple Myeloma. Cancers. 2013; 5(2):430-461.
9. Neri P, Bahlis N J, Lonial S. Panobinostat for the treatment of multiple myeloma. Expert Opin Investig Drugs. 2012; 21(5):733-747.
10. Neri P, Tagliaferri P, Di Martino M T, et al. In vivo anti-myeloma activity and modulation of gene expression profile induced by valproic acid, a histone deacetylase inhibitor. Br J Haematol. 2008; 143(4):520-531.
11. Minami J, Suzuki R, Mazitschek R, et al. Histone deacetylase 3 as a novel therapeutic target in multiple myeloma. Leukemia. 2013.
12. Hideshima T, Mazitschek R, Santo L, et al. Induction of differential apoptotic pathways in multiple myeloma cells by class-selective histone deacetylase inhibitors. Leukemia. 2013.
13. Zhang Q L, Wang L, Zhang Y W, et al. The proteasome inhibitor bortezomib interacts synergistically with the histone deacetylase inhibitor suberoylanilide hydroxamic acid to induce T-leukemia/lymphoma cells apoptosis. Leukemia. 2009; 23(8):1507-1514.
14. Richardson P G, Schlossman R L, Alsina M, et al. PANORAMA 2: panobinostat in combination with bortezomib and dexamethasone in patients with relapsed and bortezomib-refractory myeloma. Blood. 2013; 122(14):2331-2337.
15. Dimopoulos M, Siegel D S, Lonial S, et al. Vorinostat or placebo in combination with bortezomib in patients with multiple myeloma (VANTAGE 088): a multicentre, randomised, double-blind study. Lancet Oncol. 2013; 14(11):1129-1140.
16. San-Miguel J F, Hungria V T, Yoon S S, et al. Panobinostat plus bortezomib and dexamethasone versus placebo plus bortezomib and dexamethasone in patients with relapsed or relapsed and refractory multiple myeloma: a multicentre, randomised, double-blind phase 3 trial. Lancet Oncol. 2014; 15(11):1195-1206.
17. Moreaux J, Reme T, Leonard W, et al. Gene expression-based prediction of myeloma cell sensitivity to histone deacetylase inhibitors. Br J Cancer. 2013; 109(3):676-685.
18. Moreaux J, Reme T, Leonard W, et al. Development of gene expression-based score to predict sensitivity of multiple myeloma cells to DNA methylation inhibitors. Mol Cancer Ther. 2012; 11(12):2685-2692.
19. Beck D B, Oda H, Shen S S, Reinberg D. PR-Set7 and H4K20me1: at the crossroads of genome integrity, cell cycle, chromosome condensation, and transcription. Genes Dev. 2012; 26(4):325-337.
20. Brustel J, Tardat M, Kirsh O, Grimaud C, Julien E. Coupling mitosis to DNA replication: the emerging role of the histone H4-lysine 20 methyltransferase PR-Set7. Trends Cell Biol. 2011; 21(8):452-460.
21. Jorgensen S, Schotta G, Sorensen C S. Histone H4 lysine 20 methylation: key player in epigenetic regulation of genomic integrity. Nucleic Acids Res. 2013; 41(5):2797-2806.
22. Shi X, Kachirskaia I, Yamaguchi H, et al. Modulation of p53 function by SET8-mediated methylation at lysine 382. Molecular cell. 2007; 27(4):636-646.
23. Dhami G K, Liu H, Galka M, et al. Dynamic methylation of Numb by Set8 regulates its binding to p53 and apoptosis. Molecular cell. 2013; 50(4):565-576.
24. Takawa M, Cho H S, Hayami S, et al. Histone lysine methyltransferase SETD8 promotes carcinogenesis by deregulating PCNA expression. Cancer Res. 2012; 72(13):3217-3227.
25. Koturbash I, Simpson N E, Beland F A, Pogribny I P. Alterations in histone H4 lysine 20 methylation: implications for cancer detection and prevention. Antioxid Redox Signal. 2012; 17(2):365-374.
26. Hose D, Reme T, Hielscher T, et al. Proliferation is a central independent prognostic factor and target for personalized and risk-adapted treatment in multiple myeloma. Haematologica. 2011; 96(1):87-95.
27. De Vos J, Thykjaer T, Tarte K, et al. Comparison of gene expression profiling between malignant and normal plasma cells with oligonucleotide arrays. Oncogene. 2002; 21(44):6848-6857.
28. Barlogie B, Tricot G, Rasmussen E, et al. Total therapy 2 without thalidomide in comparison with total therapy 1: role of intensified induction and posttransplantation consolidation therapies. Blood. 2006; 107(7):2633-2638.
29. Kassambara A, Hose D, Moreaux J, et al. Genes with a spike expression are clustered in chromosome (sub)bands and spike (sub)bands have a powerful prognostic value in patients with multiple myeloma. Haematologica. 2012; 97(4):622-630.
30. Xiong W, Wu X, Starnes S, et al. An analysis of the clinical and biologic significance of TP53 loss and the identification of potential novel transcriptional targets of TP53 in multiple myeloma. Blood. 2008; 112(10):4235-4246.
31. Jourdan M, Caraux A, De Vos J, et al. An in vitro model of differentiation of memory B cells into plasmablasts and plasma cells including detailed phenotypic and molecular characterization. Blood. 2009; 114(25):5173-5181.
32. Jourdan M, Caraux A, Caron G, et al. Characterization of a transitional preplasmablast population in the process of human B cell to plasma cell differentiation. J Immunol. 2011; 187(8):3931-3941.
33. Ma A, Yu W, Li F, et al. Discovery of a selective, substrate-competitive inhibitor of the lysine methyltransferase SETD8. J Med Chem. 2014; 57(15):6822-6833.
34. Moreaux J, Bruyer A, Veyrune J L, Goldschmidt H, Hose D, Klein B. DNA methylation score is predictive of myeloma cell sensitivity to 5-azacitidine. Br J Haematol. 2014; 164(4):613-616.
35. Mahtouk K, Jourdan M, De Vos J, et al. An inhibitor of the EGF receptor family blocks myeloma cell growth factor activity of HB-EGF and potentiates dexamethasone or anti-IL-6 antibody-induced apoptosis. Blood. 2004; 103(5):1829-1837.
36. Cui X, Churchill G A. Statistical tests for differential expression in cDNA microarray experiments. Genome Biol. 2003; 4(4):210.
37. Subramanian A, Tamayo P, Mootha V K, et al. Gene set enrichment analysis: a knowledge-based approach for interpreting genome-wide expression profiles. Proc Natl Acad Sci USA. 2005; 102(43):15545-15550.
38. Zhan F, Huang Y, Colla S, et al. The molecular classification of multiple myeloma. Blood. 2006; 108(6):2020-2028.
39. Moreaux J, Klein B, Bataille R, et al. A high-risk signature for patients with multiple myeloma established from the molecular classification of human myeloma cell lines. Haematologica. 2011; 96(4):574-582.

1. A method of treating multiple myeloma in a patient in need thereof comprising administering to the patient a therapeutically effective amount of a SETD8 inhibitor.

2. The method of claim 1 wherein the SETD8 inhibitor is:

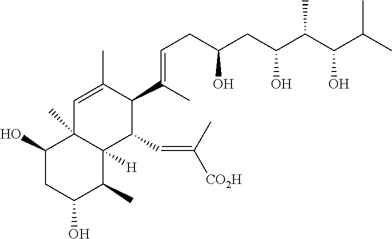 [SMILES: CC1=C[C@@]2(C)[C@H]([C@@H](C)[C@H](O)C[C@H]2O)[C@@H](/C=C(\C)C(=O)O)[C@@H]1/C(C)=C/C[C@H](O)C[C@@H](O)[C@H](C)[C@@H](O)C(C)C]

3. The method of claim 1 wherein the SETD8 inhibitor is:

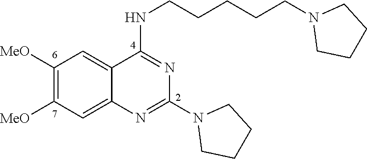 [SMILES: COc1cc2nc(N3CCCC3)nc(NCCCCCN3CCCC3)c2cc1OC]

4. The method of claim 1 wherein the SETD8 inhibitor is an inhibitor of SETD8 expression.

5. The method of claim 1 wherein the SETD8 inhibitor is administered to the patient in combination with at least one chemotherapeutic agent.

6. The method of claim 5 wherein the chemotherapeutic agent is selected from the group consisting of alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azascrine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycins, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; frolinic acid; aceglatone; aldophosphamide glycoside; aminolevulinic acid; amsacrine; bestrabucil; bisantrene; edatraxate; defo famine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′2″-trichlorotriethylamine; vindesine; dacarbazine; mannomustine; mitobronitol; mito lactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; etoposide (VP-16); ifosfamide; mitomycin C; mitoxantrone; vinblastine; vincristine; vinorelbine; navelbine; novantrone; teniposide; daunomycin; aminopterin; xeloda; ibandronate; difluoromethylornithine (DMFO); retinoic acid; esperamicins; capecitabine; imexon; tyrosine kinase inhibitors, such as epidermal growth factor receptor tyrosine kinase inhibitor erlotinib; proteasome inhibitors such as bortezomib thalidomide, lenalidomide corticosteroids such as prednisone and dexamethasone (Decadron®). pomalidomide, the keto-epoxide tetrapeptide proteasome carfilzomib, the anti-CS-1 antibody elotuzumab, and histone deacetylase inhibitors of vorinostat and panabinostatand pharmaceutically acceptable salts, acids or derivatives of any of the above.

7. A method for predicting the survival time of a patient suffering from multiple myeloma comprising i) determining the expression level of SETD8 in a sample of multiple myeloma cells obtained from the patient, ii) comparing the expression level determined at step i) with a predetermined reference value and iii) concluding that the patient will have a short survival time when the level determined at step i) is higher than the predetermined reference value or concluding that the patient will have a long survival time when the expression level determined at step i) is lower than the predetermined reference value.

8. A method of treating multiple myeloma in a subject in need thereof comprising i) determining the survival time of the patient by the method of claim 7 and ii) administering to the patient a therapeutically effective amount of a SETD8 inhibitor when it is concluded that the patient will have a short survival time.
